(19) United States
(12) Patent Application Publication
(10) Pub. No.: US 2006/0241527 A1
(43) Pub. Date: Oct. 26, 2006

(54) System and method for inducing controlled cardiac damage

(76) Inventors: Robert Muratore, Huntington, NY (US); Shunichi Homma, New York, NY (US); Daniel Burkhoff, West Harrison, NY (US); Jie Wang, Englewood Cliffs, NJ (US)

Correspondence Address: BAKER & BOTTS, 30 ROCKEFELLER PLAZA, 44TH FLOOR, NEW YORK, NY, 10112, US

(21) Appl. No.: 11/366,357

(22) Filed: Mar. 2, 2006

Related U.S. Application Data
(60) Provisional application No. 60/658,351, filed on Mar. 3, 2005.

(51) Int. Cl. A61H 1/00; A61B 8/00

(52) U.S. Cl. 601/2; 600/439

(57) ABSTRACT

A murine myocardial infarction model is provided. Cardiac damage or coronary defects are induced in the model by non-invasive application of focused high intensity ultrasound energy. The size or extent of the defects is controlled by varying ablation time, exposure number, pulse repetition rate, and acoustic intensity.

### CROSS-REFERENCE TO RELATED APPLICATIONS

This application claims the benefit of U.S. provisional patent application No. 60/658,351 filed on Mar. 3, 2005, which application is hereby incorporated by reference herein in its entirety.

Portions of this research were supported by research grant RO1 CA84588 awarded by the National Cancer Institute and the National Heart, Lung, and Blood Institute.

### FIELD OF THE INVENTION

The present invention relates generally to systems and methods of biomedical and genetic science. The invention in particular relates to animal models that are used in biomedical and genetic research.

### BACKGROUND OF THE INVENTION

Animal testing (also referred to as animal research) refers to the use of non-human animals in experiments. Animal experiments are carried out, for example, for basic or pure research, studying diseases and developing medicines, and toxicology testing of chemicals. The testing is carried out inside universities, medical schools, pharmaceutical companies, commercial facilities that provide animal-testing services to industry, on farms, in defense-research establishments, and by public-health authorities, on a variety of species from fruit flies and mice to non-human primates.

The particular species selected for biomedical testing is often based on a suitable animal model of the biological phenomena or disease under investigation. Animal model refers to a non-human animal with a disease that is similar to a human condition. Mice are convenient in research because their physiology is similar to that of humans and their short life cycle makes breeding easy. They are mainly used to model human diseases in order to develop new drugs, to test the safety of proposed drugs, and in basic research.

In order to serve as a useful model, a modeled disease must be similar in etiology (mechanism of cause) and function to the human equivalent. Animal models are used to learn more about a disease, its diagnosis and its treatment. For example, the murine model (i.e., mouse) is an important animal model for studying the cardiovascular system. The murine myocardial infarction model is widely used as an ischemic heart model and a heart failure model. Gene-targeted mouse models have been extensively used for the research on cardiovascular diseases and for understanding the molecular mechanism of heart failure.

Animal models of disease can be spontaneous, or be induced by physical, chemical or biological means. The murine myocardial infarction model is generally induced by surgical ligation of the proximal left anterior descending coronary artery. However, opening the thoracic cavity, which is necessary for this purpose, may lead to infection and death. Further, the surgical ligation technique does not provide good control of the degree of the resulting myocardial damage.

Consideration is now directed towards improving the murine model for cardiac disease investigations. In particular, attention is directed to inducing coronary defects and cardiac failure in the murine (mouse) model.

### SUMMARY OF THE INVENTION

A device and method is provided for inducing coronary defects and cardiac failure in the murine model. The device is configured to generate high intensity ultrasound waves, which are focused on a subject mouse to ablate cardiac tissue in-vivo and to cause cardiac damage. High intensity focused ultrasound (HIFU) produces immediate focal lesions with ultrasound exposures within short periods. Useful murine myocardial failure models may be created using HIFU.

The HIFU technique is a noninvasive extracorporeal technique capable of ablating subsurface structures without injuring intervening tissues. Ultrasonic energy can be applied in a target volume to induce tissue necrosis. The HIFU technique has an advantage over other ablative techniques because the tissue in the acoustic focal volume during HIFU ablation is rapidly damaged by a remote energy source (the ultrasonic transducer), and the intervening tissue is not damaged.

The HIFU technique can be used for targeted LV wall thinning, LV dilatation and systolic dysfunction in animals without thoracotomy. HIFU may be used to nonivasively create murine or other animal myocardial failure models.

The HIFU technique may be modified or extended to alternately or additionally use hyperthermia from ultrasound and other heat sources, other focused ultrasound ablation technologies such as tissue emulsification, and other ablation technologies such as ethanol injection for inducing coronary defects and cardiac failure.

### BRIEF DESCRIPTION OF THE DRAWINGS

Further objects and advantages of the invention will be apparent from a reading of the following description in conjunction with the accompanying drawings in which:

FIG. 1A is an illustration of the High Intensity Focused Ultrasound (HIFU) transducer device, which can be used to induce cardiac defects in accordance with the principles of the present invention.

FIG. 1B is an illustration of the focal zone beam shape of the output of the HIFU transducer of FIG. 1A. The output is measured using a pulse-echo reciprocity technique with a point target.

FIG. 2 is an illustration of the HIFU transducer surface (FIG. 1A) coupled with the intercostals muscle of a mouse using gel and water baths, in accordance with the principles of the present invention.

FIG. 3 shows hematoxylin and eosin stains and Masson's trichrome stains of the transverse left ventricle (LV) middle sections from an exemplary group of mice (“the HIFU group”) treated in accordance with the principles of the present invention.

FIG. 4 is a table showing the weight of the body, heart, lung and liver of the HIFU group as compared to the control group.

FIG. 5 is a table showing the LV diameter at end-diastole phase (end diastolic dimension, EDD) and at end-systole phase (end systolic dimension, ESD), and fractional shortening (FS) transthoracically for the HIFU group and control group before and after ablation, in accordance with the principles of the present invention.

FIG. 6 is an illustration of a sample PXI implementation of an ultrasound therapy system for supplying the therapeutic ultrasound energy, in accordance with the principles of the present invention.

FIG. 7 is a block diagram of an example of a simple gate mechanism for an ultrasound therapy system, in accordance with the principles of the present invention.

FIG. 8 is an example of a simple embodiment of an ultrasound therapy system for supplying the therapeutic ultrasound energy, in accordance with the principles of the present invention.

### DESCRIPTION OF THE INVENTION

Coronary defects and cardiac failure are obtained in an animal model by ablating cardiac tissue using focused ultrasound energy such as HIFU. An ablation device, which includes a high intensity ultrasound transducer, is used to generate and focus ultrasound energy on a subject heart. The focused high intensity ultrasound waves ablate cardiac tissue in localized regions and accordingly, or otherwise, cause coronary defects and cardiac failure.

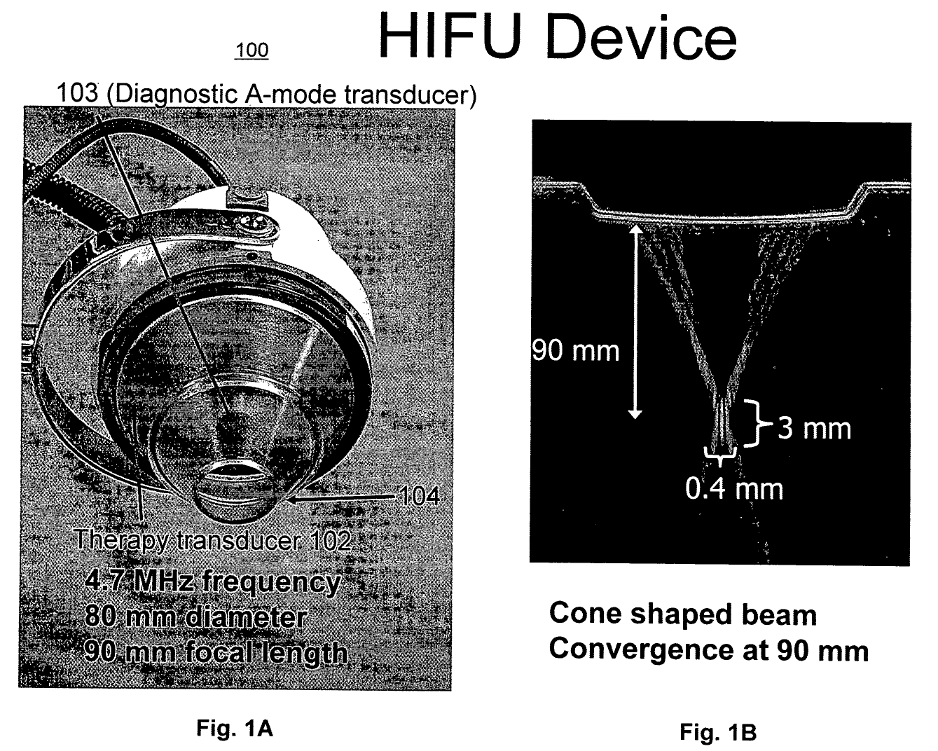

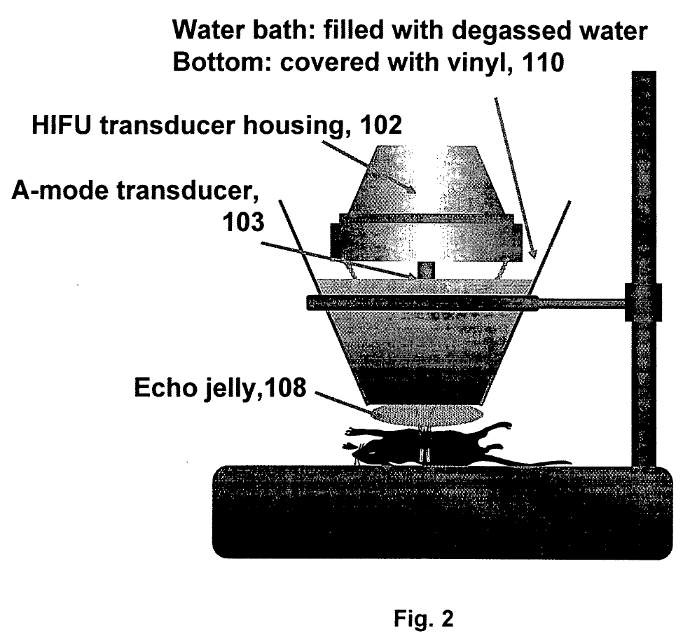

FIGS. 1A and 2 show an exemplary ablation device 100. Ablation device 100 includes a therapeutic focused ultrasound transducer 102, which produces high intensity ultrasound waves. In the exemplary device, transducer 102 is specified to produce ultrasound waves of about 4.7 MHz focused at 90 mm, with a half-power focal region approximately 3 mm axial and 0.4 mm transverse to the beam. Cone 104 is designed to contain water for coupling. (See FIG. 1B).

In one version of operation, the transducer is acoustically coupled with the intercostals muscle of a subject mouse 106 using a water path through cone 102 and bath 110, and through echo gel 108. (FIG. 2).

In one embodiment, high intensity focused ultrasound (HIFU) produces rapid focal lesions. Useful myocardial failure animal models may be created using HIFU. The HIFU technique is capable of producing transmural myocardial injury on animal hearts, resulting in animal heart failure models that are created noninvasively.

Post-infarct LV remodeling is a progressive process involving LV chamber dilatation, infarcted wall thinning, fibrous change, and compensatory thickening in the non-infarcted regions.

In a study to assess the feasibility of cardiac failure model creation using HIFU, a group of 30 wild type mice was selected. The study was designed to assess the chronic lesions of murine myocardial tissue after HIFU ablation and the feasibility of a murine heart failure model induced by HIFU ablation using conventional 2-D echocardiography.

A commercial ultrasound therapy system (Model CST-100, sold by Sonocare Inc., Ridgewood, N.J.), which is originally designed for clinical glaucoma therapy, was modified for use as the source of HIFU energy for the study. The system includes a signal generator, a power amplifier, and a transducer assembly. (See FIGS. 1A, 1B, and 2). The transducer's focal length is about 90 mm (FIG. 1A). The 80-mm diameter, 90-mm focal length spherical cap PZT-4 therapy transducer has a central 23-mm hole, which houses a 7.5 MHz A-mode diagnostic transducer (Model MD 3657, sold by Panametrics, Inc. of Waltham, Mass.). The diagnostic transducer is aligned to be coaxial and confocal with the HIFU transducer. In the study, the operating frequency of the HIFU transducer was 4.7 MHz and the ultrasound energy was applied with an acoustic power of 35 W, as determined from acoustic radiation force measurements. The focal zone beam shape was measured using a pulse-echo reciprocity technique with a point target; at the half-power points the focal zone was 3 mm in depth and 0.4 mm wide (FIG. 1B).

The transducer assembly was attached to an acrylic resin coupling cone with a 25 mm diameter exit hole. The cone was filled with degassed water, and the exit hole was covered with a latex membrane. The focus of ultrasound beam was 2.5 cm distal from the membrane at the tip of the coupling cone. The focus of ultrasound beam was positioned at the desired tissue location by distance measurements made with the diagnostic A-mode transducer.

Study Protocol

The 30 wild type mice, age 6-8 weeks and ranging in body weight from 30-39 g, were housed in a facility with a 12/12 light and dark cycle, and free access to water and mouse pellets. The mice were randomly divided into two groups: a test group (20 mice) and a control group (10 mice). For each mouse in the subject groups, cardiac characteristics were measured transthoracically using a high frequency ultrasound system. The measured characteristics included, for example, the left ventricular (LV) diameter at end-diastole and end-systole phases (EDD/ESD), and ejection fraction or fractional shortening (FS).

Surgery and HIFU ablation was performed without thoracotomy on the test group of 20 mice (HIFU group). The animals in the HIFU group were first anesthetized using Isoflurane (for induction 3.0% and maintenance 1.5±2.0%), and their chests were shaved. The subject animals were intubated with a 20 G intravenous catheter through the oral cavity under visualization and ventilated with a mixture of oxygen and room air, using a rodent ventilator at a tidal volume of 2 to 4 ml and a respiratory rate of 130 to 150 per minute.

Animals in the HIFU group underwent a left midsternal skin incision through the fifth or sixth intercostal space. The skins were retracted by use of 5-0 or 6-0 silk suture. Slight rotation of the subject animals to the right oriented the heart to better expose the left ventricle (LV). Their pectoralis major muscles and pectoralis minor muscles were moved to the sides. The beating heart was visible through the nearly transparent intercostal muscle. The HIFU transducer surface was coupled with the intercostals muscle using echo gel and a water bath (FIG. 2). The depth of the heart from the chest surface was measured using the diagnostic A-mode transducer, and the therapeutic transducer focal point was set to the middle of the LV anterior wall.

Each mouse in the HIFU group was subject to three HIFU energy discharge pulses. Each pulse was about one second in duration and had a nominal spatial-peak temporal-average intensity of about 19.7 kW/cm².

A sham operation was performed on the control group of 10 mice. Animals in control group were anesthetized and underwent only intubation and skin incision without use of HIFU ablation. After these procedures, the lungs were re-expanded and the chest was closed. The control animals were taken off the respirator and allowed to recover from the anesthesia in a warm cage.

Transthoracic echocardiography was performed on all surviving animals every week after the ablation or sham operation procedure. Four weeks later all survival animals were euthanised for morphological and histological analysis.

Transthoracic echocardiography was performed in both groups using a commercial echocardiographic system (Model Sequoia, sold by Acuson Corporation of Mountain View, Cali., 94039) equipped with a 13-MHz liner array ultrasound transducer. The transducer was used at a depth setting of 2 cm to optimize resolution. This examination was performed under light anesthesia induced by intraperitoneal injection of 2,2,2-tribromoethanol (Avertin, 2.5% solution, 0.005 ml/g body weight), which produced a semiconscious state in which the animals breathed spontaneously. The animal chests were shaved and the animals were placed on a heating table in a left lateral decubitus position. Before the procedure and every week after the procedure, the following parameters were measured in the parasternal short axis view of a 2-dimensional image at the level close to the papillary muscles: LV diameter at the end-diastolic and end-systolic phases (EDD and ESD, respectively). LV fractional shortening (FS) was calculated as
FS=[(EDD−ESD)/EDD]×100%.

The statistical results for each group of animals were expressed as mean values±one standard deviation. The paired t-test was used for the comparison within each group. The unpaired t-test was used to compare the results between the HIFU and control groups. Statistical significance was defined as a p-value of less than 0.05.

Morphological and histological examinations were conducted after four weeks. Euthanasia was performed by CO₂ exposure or overdose of pentobarbital (euthanasia solution, 100 mg/kg) intraperitoneally. Animal hearts and other organs were taken out. Each heart, lung and liver weights were obtained. Each heart was fixed in 10% formalin, and cut in paraffin blocks. Standard hematoxylin and eosin (H&E) stained slides and Masson's trichrome stained slides were evaluated for pathological evidence of injury, inflammation, and scarring.

Study Results

The mortality of the HIFU group was 15%. HIFU ablation could be performed on all mice hearts. The overall survival after HIFU ablation was 85%. One animal in HIFU group died immediately after HIFU ablation as a result of a ruptured left anterior wall and two died of severe heart failure within three days after HIFU ablation. All the sham-operated mice survived throughout the study.

At four weeks after the ablation and surgery procedures, the cardiac characteristics of the mice in both the HIFU and control groups were evaluated for comparison with the pre-procedure characteristics.

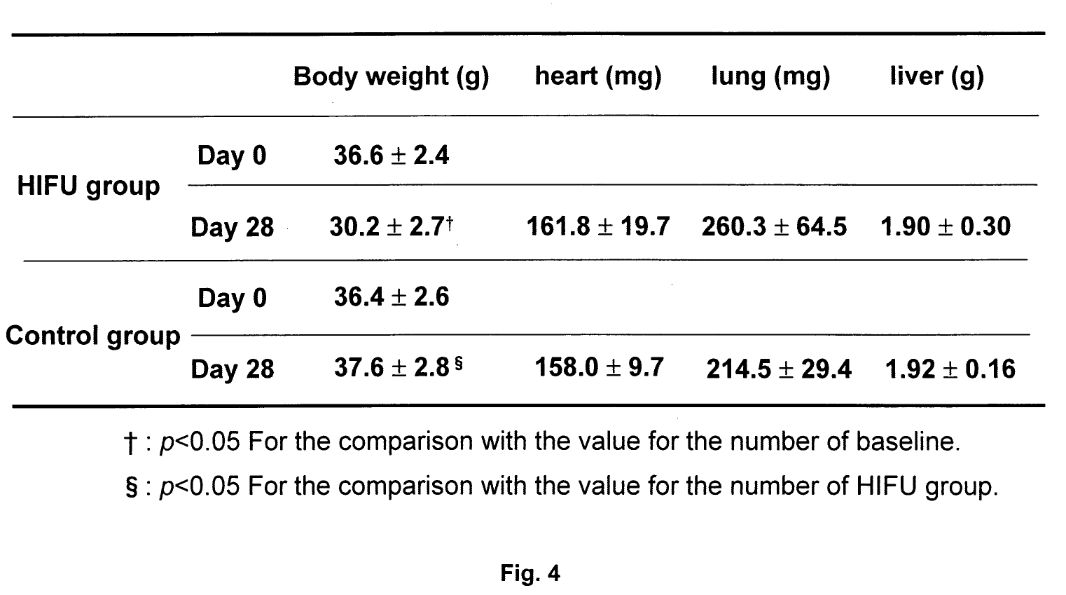

Body weight was similar in both groups before HIFU ablation (HIFU group vs. control group: 36.6±2.4 g vs. 36.4±2.6 g). In the HIFU group, body weights were significantly decreased after HIFU ablation (36.6±2.4 g vs. 30.2±2.7 g, p<0.01). The weights of whole heart and liver were not significantly different between the two groups (FIG. 4).

Technically adequate echocardiographic images were obtained in all animals for LV dimension and function measurements. However, image quality was reduced compared with echocardiographic images from non-operated animals because of residual fibrinous, exudate and fibrous adhesions.

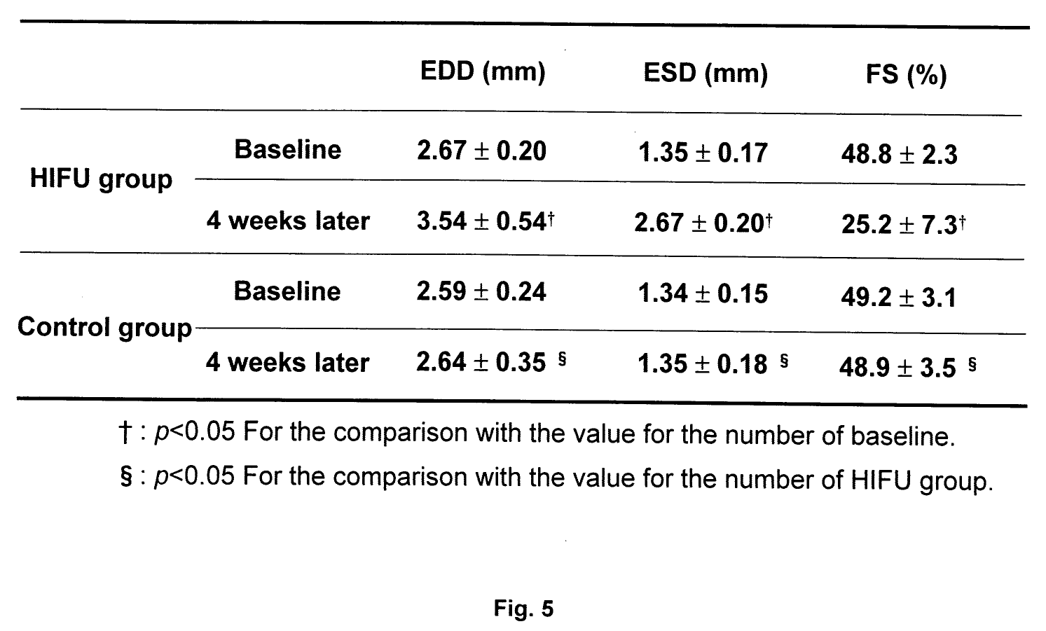

The pre-procedure LV EDD/ESD for the control mice were measured to be 1.34±0.15/2.59±0.24 mm (FIG. 5). Similarly, the pre-procedure EDD/ESD for the HIFU group were measured to be 1.35±0.17/2.67±0.2. Thus, there was no significant difference in EDD/ESD between the two groups before the HIFU ablation procedure. However, after four weeks, the treated group of mice showed considerably larger LV diameters. The post-procedure LV EDD/ESD for the HIFU group were measured to be 2.53±0.54/3.54±0.54. The pre-procedure LV ejection fraction or fractional shortening (FS) in the control group and the HIFU group of mice was similar. The post-procedure FS in the control mice did not change significantly. However, FS in the HIFU group was significantly reduced after HIFU ablation. FS in the HIFU group was measured before and after ablation to be about 48.8±2.3% and 25.2±7.3%, respectively, p<0.01.

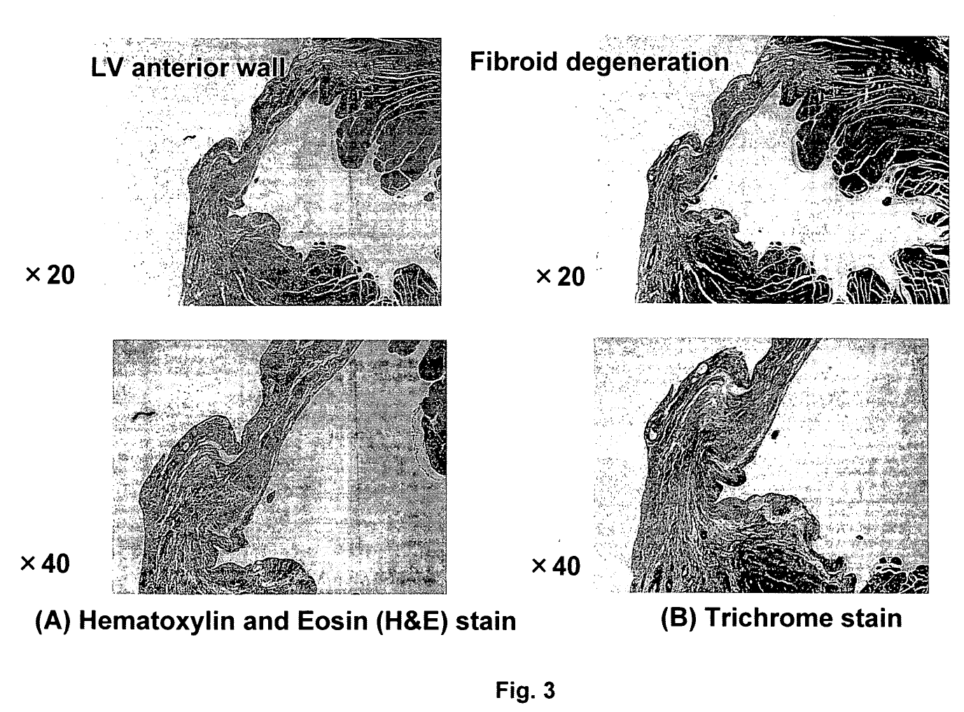

Histopathological analysis the HIFU group hearts showed necrosis (i.e., a fibroid degeneration) around the ablation site and the LV anterior wall thinning. FIG. 3 shows H&E and trichrome stains of transverse LV middle sections from the HIFU group. Myocardial injuries were identified histologically as transmural injuries in all animals of the HIFU group. At the targeted site, the myocardial tissues were changed into fibrous degeneration. LV wall thinning and LV chamber enlargements were found. The histological findings show typical characteristics of myocardial infarction. The results of the study demonstrate that HIFU can produce LV dilatation and systolic dysfunction in mice. Thus, HIFU may be used to create a murine myocardial failure model. The murine myocardial failure model with focal myocardial dysfunction is created without opening murine chests.

Using HIFU may be superior to the use of the other techniques that are used to create the murine heart failure mode. For example, in the previous studies using LAD ligation, a mortality rate in the range of 11% to 46% with an average of 27% has been observed. The mortality rate of HIFU treated mice in the above-described study was 15%. Thus, HIFU has a potential to make a murine heart failure model with minimum invasion and a high success rate.

The advantages of using HIFU may stem from its capability of producing lesions not only thermally but also through cavitation, acoustic streaming, and shear stresses. Further, the focusing ability of HIFU makes it superior to other ablative techniques such as radio frequency (RF) ablation. RF ablation and focused ultrasound ablation produce lesions with similar histological injury in myocardial tissue. However, RF is not focused and RF energy is absorbed proportionally by the distance between the tissue and the RF catheter. In contrast, ultrasound energy can be focused, allowing smaller and more precise lesions to be created.

In-vitro study shows that the eventual size of the HIFU lesion in the myocardial tissue depends on many factors. The extent of HIFU induced tissue injury and coagulative necrosis varies linearly with ablation time, exposure number, and acoustic intensity. By changing these factors smaller or larger lesions may be produced at will.

The foregoing merely illustrates the principles of the invention. It will be appreciated that those skilled in the art will be able to devise numerous modifications which, although not explicitly described herein, embody the principles of the invention and are thus within the spirit and scope of the invention. For example, it will be readily understood by those skilled in the art that by changing the focal depth or the location of the focal point, focused ultrasound ablation may be obtained at any suitable subsurface tissue. Focusing in the papillary muscle may be used to create a papillary muscle failure model without thoracotomy. Further, in the study described herein, an A-mode transducer is mounted in the center the HIFU therapy transducer for the measurement of the distance between the heart and the transducer. If a 2-D transducer is mounted in the center of the HIFU therapy transducer instead of the A-mode transducer, it may be possible to focus in the LV anterior wall and perform HIFU ablation from outside the body without skin incision. Further, for example, the HIFU technique may be utilized to create suitable disease models in other animal species (e.g., canine models). Finally, the ultrasound frequencies can be varied over a wide range to activate different defect generation mechanisms. When the ultrasound frequencies are in the range of several hundred kHz, and the ultrasound is pulsed at varying rates, tissue emulsification due to cavitation will dominate the ablation mechanism for defect generation. Similarly, when the ultrasound frequencies are in the MHz range, thermal necrosis will dominate the ablation mechanism.

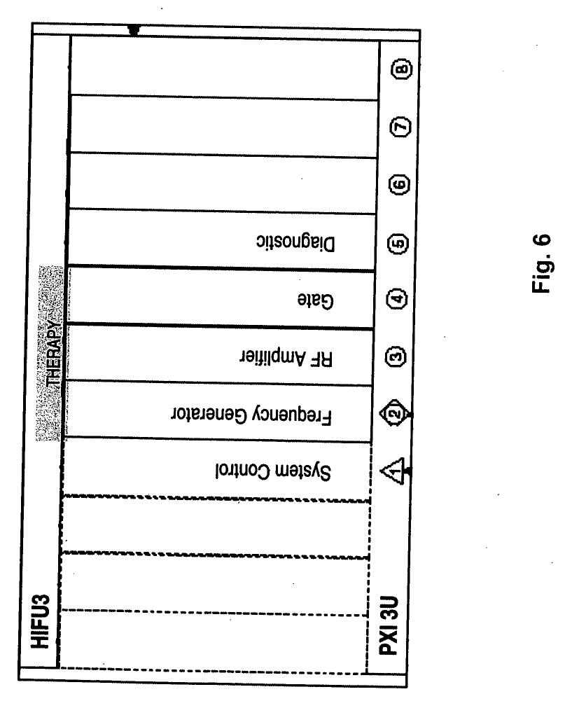

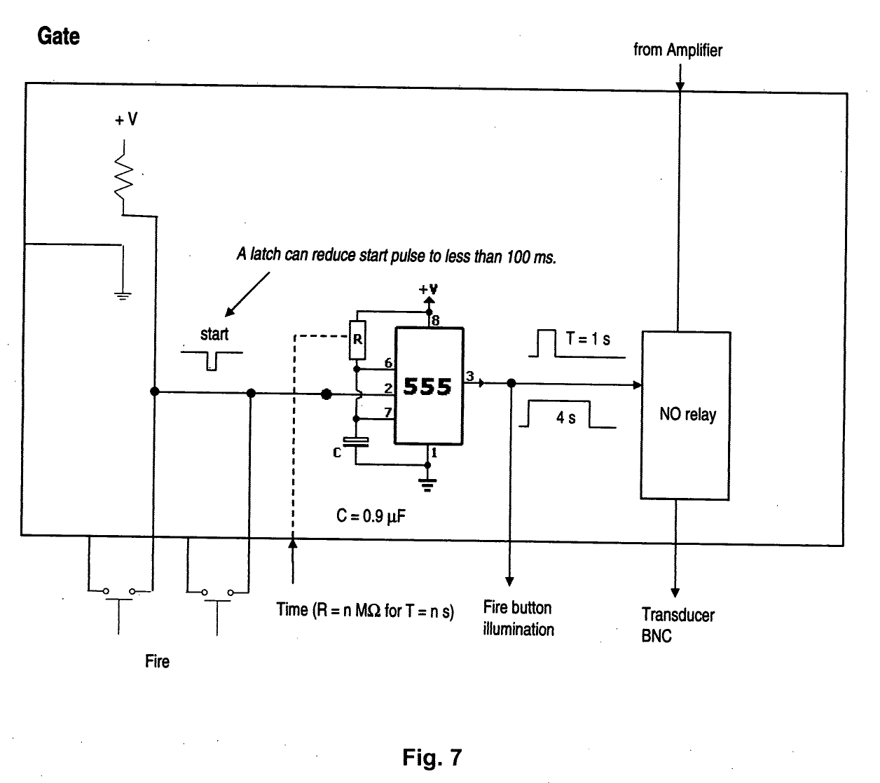

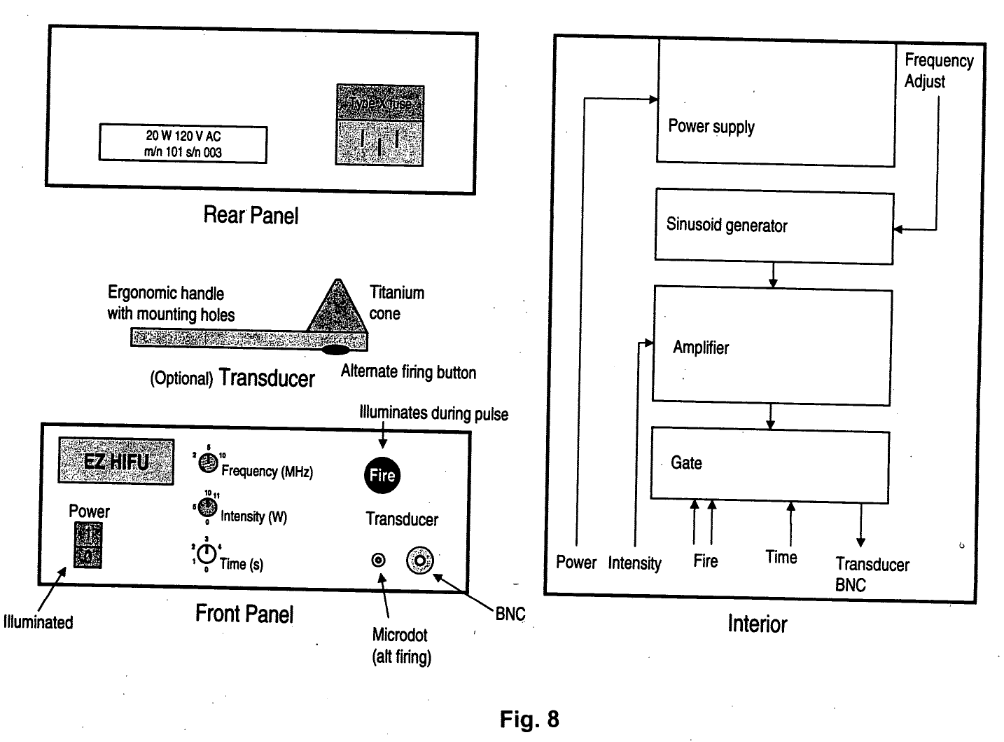

Simple low cost ultrasound systems may be used for HIFU application. FIG. 8 shows an exemplary ultrasound therapy system that can be used for HIFU application. FIG. 6 shows an exemplary PXI implementation of the ultrasound therapy system. Further, FIG. 7 shows an example of a simple gate mechanism that may be used in the ultrasound therapy system.

1. A method for preparing an animal disease model, the method comprising:
using an ultrasound generator to generate ultrasound energy;
focusing the ultrasound energy; and
exposing selected tissue regions in an animal to the focused ultrasound energy to cause tissue damage, wherein the tissue damage corresponds to an animal disease condition.

2. The method of claim 1 wherein using an ultrasound generator to generate ultrasound energy comprises extracorporeal generation of ultrasound energy.

3. The method of claim 1 wherein exposing selected tissue regions in an animal to the focused ultrasound energy comprises exposing subsurface tissue regions in the animal to the focused ultrasound energy.

4. The method of claim 1 wherein exposing selected tissue regions in an animal to the focused ultrasound energy comprises making a skin incision to expose the left ventricle (LV) of the animal.

5. The method of claim 4 wherein focusing the ultrasound energy comprises focusing the ultrasound energy at the about the middle of the LV anterior wall.

6. The method of claim 4 wherein focusing the ultrasound energy comprises measuring the depth of the heart from the chest surface using a diagnostic A-mode transducer.

7. The method of claim 1 wherein focusing the ultrasound energy comprises measuring the depth of the heart of the animal from its chest surface using a 2-D transducer.

8. The method of claim 4 wherein focusing the ultrasound energy comprises measuring the depth of the heart from the chest surface using a 2-D transducer.

9. The method of claim 1 wherein exposing selected tissue regions in an animal to the focused ultrasound energy to cause tissue damage, comprises exposing myocardial tissue.

10. The method of claim 1 wherein exposing selected tissue regions in an animal to the focused ultrasound energy to cause tissue damage, comprises exposing papillary tissue.

11. The method of claim 1 wherein exposing selected tissue regions in an animal to the focused ultrasound energy to cause tissue damage, comprises varying at least one of an ablation time, an exposure number, a pulse repetition rate, and an acoustic intensity to control tissue damage.

12. The method of claim 1 wherein exposing selected tissue regions in an animal to the focused ultrasound energy to cause tissue damage, comprises exposing the selected tissue regions in the animal to ultrasound energy having a frequency in the kHz or MHz ranges.

13. The method of claim 1 wherein the animal is a murine or canine species.

14. A system for preparing an animal disease model, the system comprising:
an ultrasound energy generator; and
a focusing arrangement for focusing the generated ultrasound energy on selected tissue regions in an animal,
wherein the focused ultrasound energy intensity is such that tissue damage is caused in the exposed regions, and wherein the tissue damage corresponds to an animal disease condition.

15. The system of claim 14 wherein the ultrasound energy generator comprises a transducer for measuring the depth of the selected tissue regions from the body surface of the animal.

16. A murine myocardial infarction model wherein cardiac defects are induced by application of ultrasound energy.
